(19) United States
(12) Patent Application Publication
(10) Pub. No.: US 2010/0069319 A1
(43) Pub. Date: Mar. 18, 2010

(54) Mercaptopurine derivatives and uses thereof

(76) Inventors: Talia Miron, Kfar Haim (IL); Aharon Rabinkov, Rehovot (IL); David Mirelman, Ramat Efal (IL); Meir Wilchek, Rehovot (IL)

Correspondence Address: BROWDY AND NEIMARK, P.L.L.C.;624 NINTH STREET, NW, SUITE 300, WASHINGTON, DC, 20001-5303, US

(21) Appl. No.: 12/529,706

(22) PCT Filed: Mar. 2, 2008
(86) PCT No.: PCT/IL08/00268

§ 371 Date: Sep. 2, 2009

Related U.S. Application Data
(60) Provisional application No. 60/904,405, filed on Mar. 2, 2007.

(51) Int. Cl. A61K 31/70; C07D 473/00; A61K 31/52; C07H 19/167

(52) U.S. Cl. 514/45; 544/264; 514/263.1; 536/27.8

(57) ABSTRACT

The invention provides novel mercaptopurine derivatives, e.g., S-allylthio-6-mercaptopurine and S-allylthio-6-mercaptopurine 9-riboside, as well as pharmaceutical compositions thereof. These compounds are highly efficient anti-proliferative agents, thus can be useful for treatment of various diseases or disorders, in particular, proliferative, inflammatory, skin and immune diseases or disorders.

### FIELD OF THE INVENTION

The present invention relates to novel mercaptopurine derivatives and to pharmaceutical compositions thereof. The mercaptopurine derivatives are useful for treatment of various diseases or disorders, in particular, proliferative, inflammatory, skin and autoimmune diseases or disorders.

### BACKGROUND OF THE INVENTION

6-Mercaptopurine (6-MP), first synthesized by Elion et al. (1952), as well as metabolically related compounds thereof such as azathioprine, 6-mercaptopurine riboside (6-MPR, also known as thioinosine), 6-thiouric acid, 6-methylmercaptopurine (6-MMP), 6-methyl-thioinosine 5′-monophosphate, and 6-thioguanine (6-TG), are structural analogs of adenine and guanine, thus the therapeutic and, in particular, antiproliferative properties of this class of compounds have long been recognized.

In fact, 6-MP and 6-MPR are cytotoxic prodrugs that interfere with nucleic acid synthesis by either direct substitution of deoxythioGTP, thereby causing further modifications and mismatches upon replication, or by inhibition of de novo purine biosynthesis (Karran, 2006).

Of these compounds, 6-MP, azathioprine and 6-MPR are the most prominent therapeutic agents, and as such are currently used in the treatment of a variety of medical conditions such as cancer (in particular leukemia), inflammatory bowel diseases (in particular Crohn's disease and ulcerative colitis), psoriatic arthritis, psoriasis, Reiter's syndrome, Behcet's disease, polymyositis, systemic lupus erythematosus and systemic vasculitis. Azathioprine is further used in the prevention of rejection following organ transplants (Carroll et al., 2003; Watters and McLeod, 2003; Dubinsky, 2004).

Although various analogs of mercaptopurine have been devised, they suffer major therapeutic disadvantages, particularly dose limiting toxicity. In particular, treatments involving administration of these analogs are often associated with adverse side effects, such as a high incidence of birth defects and, when used for prolonged time periods, bone marrow depression, liver damage, drug-induced pneumonia and pancreatitis.

Thiopurines are prodrugs that are transformed enzymatically by three competitive enzymatic pathways: the first, xanthine oxidase, catalyzes the oxidation of 6-MP to the biologically inactive metabolite, thiouric acid; the second, hypoxanthine-guanine phosphoribosyl transferase (HGPRT), catalyzes the formation of 6-thioinosine monophosphate that may further be converted by cellular enzymes to thioguanine nucleotides that may then be incorporated by polymerase directly into the DNA; and the third, thiopurine methyltransferase (TPMT), catalyzes S-methylation of 6-MP and 6-thioinosine monophosphate to 6-methyl-mercaptopurine (MeMP) and S-methyl-thioinosine 5′-monophosphate (MeTIMP), respectively. The latter is a potent inhibitor of phosphoribosylpyrophosphate amidotransferase, the first step of de novo purine biosynthesis, thereby causing purine depletion (Karran, 2006; Coulthard and Hogarth, 2005; Krynetski and Evans, 1999; Cara, et al., 2004).

Hence, while 6-MP, 6-MPR and various derivatives thereof have exceptional therapeutic potential, particularly in the fields of proliferative and inflammatory diseases and disorders, their practice is often limited, mainly due to cytotoxicity and/or poor pharmacokinetic thereof, as described hereinabove. Thus, there is a widely recognized need for, and it would be highly advantageous to have, novel therapeutic agents which would possess the biological activities of mercaptopurines, and at the same time would circumvent the limitations associated with the use of these compounds in various treatments.

### SUMMARY OF THE INVENTION

It has been found in accordance with the present invention that mercaptopurine derivatives, e.g., S-allylthio-6-mercaptopurine and S-allylthio-6-mercaptopurine 9-riboside, are highly efficient anti-proliferative agents, having DNA synthesis inhibitory effect that is either equal or superior to that of 6-mercaptopurine and 6-mercaptopurine riboside, respectively, while further circumventing the limitations associated with the administration of each of the latter alone.

In one aspect, the present invention thus relates to a compound of the general formula

X—S—S—Y

wherein X is a purine residue of the general formula:

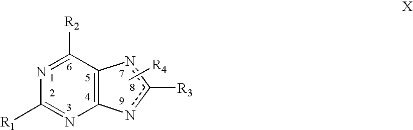

wherein

R₁ to R₃ each independently is either a covalent bond or selected from H, halogen, SH, NR₅R₆, O-hydrocarbyl, S-hydrocarbyl, heteroaryl, unsubstituted hydrocarbyl, hydrocarbyl substituted by halogen, CN, SCN, NO₂, OR₅, SR₅, NR₅R₆ or heteroaryl, or a carbohydrate residue, wherein R₅ and R₆ each independently is H or hydrocarbyl or R₅ and R₆ together with the nitrogen atom to which they are attached form a 5- or 6-membered saturated heterocyclic ring optionally containing 1-2 further heteroatoms selected from the group consisting of oxygen, nitrogen and sulfur, the additional nitrogen being unsubstituted or substituted by alkyl substituted by halogen, hydroxyl or phenyl, provided that one of R₁, R₂ and R₃ is a covalent bond;

R₄ is H, alkyl, a carbohydrate residue or NR₅R₆, wherein R₅ and R₆ each independently is H or hydrocarbyl or R₅ and R₆ together with the nitrogen atom to which they are attached form a 5- or 6-membered saturated heterocyclic ring optionally containing 1-2 further heteroatoms selected from the group consisting of oxygen, nitrogen and sulfur, the additional nitrogen being unsubstituted or substituted by alkyl substituted by halogen, hydroxyl or phenyl;

Y is heteroaryl, unsubstituted hydrocarbyl, or hydrocarbyl substituted by halogen, CN, SCN, NO₂, OR₇, SR₇, NR₇R₈ or heteroaryl, wherein R₇ and R₈ each independently is H or hydrocarbyl or R₇ and R₈ together with the nitrogen atom to which they are attached form a 5- or 6-membered saturated heterocyclic ring optionally containing 1-2 further heteroatoms selected from the group consisting of oxygen, nitrogen and sulfur, the additional nitrogen being unsubstituted or substituted by alkyl substituted by halogen, hydroxyl or phenyl; and

the dashed line denotes a double bond between the carbon atom at position 8 and either the nitrogen atom at position 7 or the nitrogen atom at position 9, provided that, when the double bond is between the carbon atom at position 8 and the nitrogen atom at position 7, R₄ is at position 9, and when the double bond is between the carbon atom at position 8 and the nitrogen atom at position 9, R₄ is at position 7;

and pharmaceutically acceptable salts thereof.

In another aspect, the present invention relates to a pharmaceutical composition comprising a compound as defined above, or a pharmaceutically acceptable salt thereof, and a pharmaceutical acceptable carrier.

The compounds and the pharmaceutical compositions of the present invention may be used for treatment of a disease or disorder selected from a proliferative disease or disorder, an inflammatory disease or disorder, a skin disease or disorder, or an immune disease or disorder.

Thus, in a further aspect, the present invention relates to use of a compound as defined above, or a pharmaceutically acceptable salt thereof, for the treatment of a disease or disorder selected from a proliferative disease or disorder, an inflammatory disease or disorder, a skin disease or disorder, or an immune disease or disorder.

The present invention further provides a method for treatment of a disease or disorder selected from a proliferative disease or disorder, an inflammatory disease or disorder, a skin disease or disorder, or an immune disease or disorder, said method comprising administering to an individual in need a therapeutically effective amount of a compound as defined above, or a pharmaceutically acceptable salt thereof.

### BRIEF DESCRIPTION OF THE FIGURES

FIGS. 1A-1B show spectrum analysis of S-allylthio-6-mercaptopurine (hereinafter SA-6MP) (1A) and S-allylthio-6-mercaptopurine 9-riboside (hereinafter SA-6MPR) (1B) in ethanol. The inserts in 1A and 1B show the HPLC elution pattern of SA-6MP and SA-6MPR, respectively. Absorbance was monitored at 210 nm.

FIGS. 2A-2C show cell proliferation of Daudi cells (2A), Hela cells (2B) and N87 cell (2C), treated with different concentrations (0-200 μM of either 6MP or SA-6MP, as determined by [³H] thymidine incorporation. Non-treated cells were used as control (100%). Cells were treated for 16 h at 37° C. The values presented are the means±SEM.

FIGS. 3A-3D show the lethal effect of 6-MP and SA-6MP on MDR HT-29 cells (3A), Hela cells (3B), Daudi cells (3C) and B-CLL cells (3D). Cells were incubated with the prodrugs for 16 h at 37° C. and stained with trypan blue and propidium iodine (PI). Cells were counted after the trypan blue exclusion test and the percentage of viable cells was calculated. Alternatively, the percentage of viable PI stained cells was determined by FACS analysis. The values presented are the means±SD. Viability values that are significantly different (p<0.05) from non-treated cells (*).

FIG. 4 shows cell death assessment using PI staining. MDR HT-29 cells were cultured in the presence of either 6-MP or SA-6MP (150 μM) for 16 h at 37° C. and stained with propidium iodide (PI). Images of treated-cells were observed by phase-contrast microscopy to determine normal growth patterns (T, upper panel) and fluorescent images of the treated-cells indicated dead cells stained with PI, (F, lower panel).

FIGS. 5A-5F show Fluorescence Activated Cell Sorting (FACS) analysis of chronic lymphocytic leukemia B-cells (B-CLL) treated for 16 h at 37° C. with 50 μM of 6MP (5B), 100 μM of 6MP (5C), 150 μM of 6MP (5D), 50 μM of SA-6MP (5E) or 100 μM of SA-6MP (5F) vs. untreated cells (5A). Cells were stained with Annexin-Cy5 and analyzed by FACS. The percentage of apoptotic cells at various drug concentrations are superimposed in the upper right part of each analysis.

FIGS. 6A-6B show percentage of apoptotic cells (6A) and of dead cells (6B) in chronic lymphocytic leukemia B-cells (B-CLL) treated with different concentrations (0-150 μM) of 6-MP or SA-6MP for 16 h at 37° C. Apoptotic cells were measured using FACS analysis with annexin and cell death was measured using trypan blue test.

FIGS. 7A-7B show the effects of 6-MP, 6-MPR, SA-6MP and SA-6MPR on cell proliferation in Daudi (7A) and N87 (7B) cell lines. Cells were incubated with different concentrations (0-150 μM) of the prodrugs for 16 h at 37° C., and the anti-proliferative effect was assessed using the XTT assay. The values presented are the means±SEM.

### DETAILED DESCRIPTION OF THE INVENTION

The present invention relates, in one aspect, to a mercaptopurine derivatives of the general formula X—S—S—Y, as defined hereinabove.

As discussed hereinabove, various mercaptopurines have been shown heretofore to exert beneficial therapeutic activity, particularly as anti-proliferative agents. These mercaptopurines share common structural features such as having a purine-like skeleton and one or more free thiol groups attached thereto, e.g., to the carbon atoms in the skeleton.

The phrase “purine-like” skeleton as used herein refers to a structure composed of a pyrimidine ring and an imidazole ring fused to one another, as well as to analogs thereof. Examples of purine-like analogs include structures in which the pyrimidine ring is replaced by a pirazine, a pyridine or a phenyl ring and/or the imidazole ring is replaced by a furan ring, a pyrrole ring and the like.

The term “analog” as used herein with respect to a mercaptopurine refers to a compound which shares chemical and/or structural characteristics of a mercaptopurine and is thus capable, for example, of blocking nucleic acid synthesis in the body.

The term “derivative” describes a compound which has been subjected to a chemical modification while maintaining its main structural features. Such chemical modifications can include, for example, replacement of one or more substituents and/or one or more functional moieties.

As used herein, the term “halogen” includes fluoro, chloro, bromo, and iodo, and it is preferably chloro.

The term “hydrocarbyl” in any of the definitions of the different radicals R₁ to R₈ refers to a radical containing only carbon and hydrogen atoms that may be saturated or unsaturated, linear or branched, cyclic or acyclic, or aromatic, and includes C₁-C₂₀ alkyl, C₂-C₂₀ alkenyl, C₂-C₂₀ alkynyl, C₃-C₂₀ cycloalkyl, C₃-C₂₀ cycloalkenyl, C₆-C₁₄ aryl, (C₁-C₂₀)alkyl(C₆-C₁₄)aryl, and (C₆-C₁₄)aryl(C₁-C₂₀)alkyl.

The term “C₁-C₂₀ alkyl” typically means a straight or branched hydrocarbon radical having 1-20 carbon atoms and includes, for example, methyl, ethyl, n-propyl, isopropyl, n-butyl, sec-butyl, isobutyl, tert-butyl, n-pentyl, 2,2-dimethylpropyl, n-hexyl, n-heptyl, n-octyl, and the like. Preferred are C₁-C₆ alkyl groups, most preferably methyl and ethyl. The terms “C₂-C₂₀ alkenyl” and “C₂-C₂₀ alkynyl” typically mean straight and branched hydrocarbon radicals having 2-20 carbon atoms and 1 double or triple bond, respectively, and include ethenyl, propenyl, 3-buten-1-yl, 2-ethenylbutyl, 3-octen-1-yl, and the like, and propynyl, 2-butyn-1-yl, 3-pentyn-1-yl, and the like. C₂-C₆ alkenyl radicals are preferred. The term “C₃-C₂₀ cycloalkyl” means a cyclic or bicyclic hydrocarbyl group such as cyclopropyl, cyclobutyl, cyclopentyl, cyclohexyl, adamantyl, bicyclo[3.2.1]octyl, bicyclo[2.2.1]heptyl, and the like. The term “C₆-C₁₄ aryl” denotes a carbocyclic aromatic radical such as phenyl and naphthyl and the term “ar(C₁-C₂₀)alkyl” denotes an arylalkyl radical such as benzyl and phenetyl.

When one or more of the radicals R₁ to R₃ are O-hydrocarbyls or S-hydrocarbyls or are hydrocarbyls substituted by a OR₅ or SR₅ radical, wherein R₅ is hydrocarbyl, each one of said hydrocarbyls is preferably a C₁-C₆ alkyl, most preferably methyl or ethyl, or an aryl, most preferably phenyl, or an aralkyl, most preferably benzyl, radical.

When the radical Y is hydrocarbyl substituted by a OR₇ or SR₇ radical, wherein R₇ is hydrocarbyl, each one of said hydrocarbyls is preferably a C₂-C₈ alkenyl or alkynyl, more preferably C₂-C₆ alkenyl or alkynyl, most preferably allyl or propargyl, radical.

In the groups and NR₅R₆ and NR₇R₈, R₅ to R₈ each independently is H or hydrocarbyl as defined above or form together with the N atom to which they are attached a saturated, preferably a 5- or 6-membered, heterocyclic ring, optionally containing 1 or 2 further heteroatoms selected from nitrogen, oxygen, and sulfur. Such rings may be substituted, for example with one or two C₁-C₆ alkyl groups, or with one alkyl or hydroxyalkyl group at a second nitrogen atom of the ring, for example in a piperazine ring. Examples of radicals NR₅R₆ and NR₇R₈ include, without being limited to, amino, dimethylamino, diethylamino, ethylmethylamino, phenylmethylamino, pyrrolidino, piperidino, tetrahydropyridino, piperazino, ethylpiperazino, hydroxyethylpiperazino, morpholino, thiomorpholino, thiazolino, and the like.

The term “heteroaryl” refers to a radical derived from a mono- or poly-cyclic ring containing one to three heteroatoms selected from the group consisting of N, O and S, with unsaturation of aromatic character. Non-limiting examples of heteroaryl include pyrrolyl, furyl, thienyl, pyrazolyl, imidazolyl, oxazolyl, isoxazolyl thiazolyl, isothiazolyl, pyridyl, 1,3-benzodioxinyl, pyrazinyl, pyrimidinyl, 1,3,4-triazinyl, 1,2,3-triazinyl, 1,3,5-triazinyl, thiazinyl, quinolinyl, isoquinolinyl, benzofuryl, isobenzofuryl, indolyl, imidazo[1,2-a]pyridyl, pyrido[1,2-a]pyrimidinyl, benz-imidazolyl, benzthiazolyl, benzoxazolyl. The heteroaryl ring may be substituted. It is to be understood that when a polycyclic heteroaromatic ring is substituted, the substitution may be in the heteroring or in the carbocyclic ring.

As detailed in the background section, mercaptopurine ribosides, being analogous to the ribonucleotide building blocks of RNA, can block nucleic acid synthesis in the body. Hence, according to a preferred embodiment of the present invention, at least one of R₁-R₄ is a carbohydrate residue.

The term “carbohydrate” describes a molecule containing carbon, hydrogen and oxygen atoms. The carbohydrate can be cyclic or linear, saturated or unsaturated and substituted or unsubstituted. Preferably, the carbohydrate residue comprises one or more saccharide residues.

The phrase “saccharide residue” as used herein encompasses any residue of a sugar moiety, including monosaccharides, oligosaccharides and polysaccharides. Alternatively, the saccharide can be a saccharide derivative such as, but not limited to, glucosides, ethers, esters, acids and amino saccharides.

Monosaccharides consist of a single sugar molecule which cannot be further decomposed by hydrolysis. Examples of monosaccharides include, without limitation, pentoses such as, but not limited to, arabinose, xylose and ribose.

Oligosaccharides are chains composed of saccharide units. As commonly defined in the art and herein, oligosaccarides are composed of up to nine saccharide units. Examples of oligosaccharides include, without limitation, disaccharides such as, but not limited to, sucrose, maltose, lactose and cellobiose; trisaccharides such as, but not limited to, mannotriose, raffinose and melezitose; and tetrasaccharides such as amylopectin, Syalyl Lewis X (SiaLex) and the like.

The term “polysaccharide” as used herein refers to compounds composed of at least 10 saccharide units and up to hundreds and even thousands of monosaccharide units per molecule, which are held together by glycoside bonds and range in their molecular weights from around 5,000 and up to millions of Daltons. Examples of common polysaccharides include, but are not limited to, starch, glycogen, cellulose, gum arabic, agar and chitin.

In one embodiment, the carbohydrate residue is a saccharide residue, preferably a monosaccharide residue, more preferably, a 5- or 6-membered monosaccharide residue, most preferably a riboside residue. In most preferred embodiments, R₄ is a riboside residue, such that the purine residue according to the present invention has a structure similar to a purine ribonucleotide, i.e. the building block of RNA, and hence can interfere with RNA synthesis and exhibit anti-proliferative effect.

The purine residue according to the present invention may be linked via a disulfide bond to Y as defined above at various positions of the pyrimidine ring, namely at positions 2, 6 or 8.

In one embodiment, the purine residue is linked via a disulfide bond to Y at position 6 of the pyrimidine ring, namely R₂ is a covalent bond.

In another embodiment, the purine residue is linked via a disulfide bond to Y at position 2 of the pyrimidine ring, namely R₁ is a covalent bond.

In a further embodiment, the purine residue is linked via a disulfide bond to Y at position 8 of the mercaptopurine, namely R₃ is a covalent bond.

In preferred embodiments, R₂ is a covalent bond, R₁ is H, SH, methyl or dimethylamino, R₃ is H, SH or methyl, R₄ is H or a 5-membered monosaccharide residue, and Y is allyl or propargyl. In more preferred embodiments, R₂ is a covalent bond, R₁ and R₃ each is H, R₄ is H or a riboside residue, and Y is allyl or propargyl.

In other preferred embidiments, wherein R₁ is a covalent bond, R₂ is H, SH, methyl or dimethylamino, R₃ is H, SH or methyl, R₄ is H or a 5-membered monosaccharide residue, and Y is allyl or propargyl. In more preferred embodiments, R₁ is a covalent bond, R₂ and R₃ each is H, R₄ is H or a riboside residue, and Y is allyl or propargyl.

In further preferred embodiments, R₃ is a covalent bond, R₁ and R₂ each independently is H, SH, methyl or dimethylamino, R₄ is H or a 5-membered monosaccharide residue, and Y is allyl or propargyl. In more preferred embodiments, R₃ is a covalent bond, R₁ and R₂ each is H, R₄ is H or a riboside residue, and Y is allyl or propargyl.

In most preferred embodiments, the compounds of the present invention are derived from 6-mercaptopurines and hence are the compounds wherein R₂ is a covalent bond. Representative examples of these compounds include, without limitation, the compounds wherein R₁, R₃ and R₄ each is H, and Y is allyl, namely S-allylthio-6-mercaptopurine, which is also abbreviated herein as SA-6MP; and the compound wherein R₁ and R₃ each is H, R₄ is riboside, and Y is allyl, namely S-allylthio-6-mercaptopurine 9-riboside, which is also abbreviated herein as SA-6MPR (see Scheme 1 hereinafter).

Allicin, the biologically active compound derived from garlic, is produced upon crushing the garlic clove, thus exposing the enzyme alliinase to its substrate, alliin (S-allyl-L-cysteine sulfoxide) (Stoll and Seebeck, 1951). Allicin is known to confer many health beneficial effects, amongst which are its anti microbial, anti fungal and anti parasitic activities (Koch and Lawson, 1996), antihypertensive activity (Elkayam et al., 2000), remedial effects on cardiovascular risk factors (Eilat et al., 1995; Abramovitz et al., 1999; Gonen et al., 2005), anti-inflammatory activity (Lang et al., 2004) and anticancer activity (Koch and Lawson, 1996; Agarwal, 1996; Hirsch et al., 2000; Miron et al., 2003; Arditti et al., 2005).

Allicin is a short-lived compound, which rapidly reacts with free thiol groups and penetrates biological membranes with ease (Rabinkov et al., 1998, 2000; Miron et al., 2000), thus shows promise in affecting different metabolic pathways (Agarwal, 1996). However, since allicin is very unstable, it quickly disintegrates in the blood a few minutes after being administered both in vitro in human blood (Freeman and Kodera, 1995) and in vivo in rats (Lachmann et al., 1994), and therefore, its therapeutic effect is limited to targets close to the gastrointestinal tract.

As previously disclosed, allicin derivatives such as allylmercaptoglutathione (GSSA) (Miron et al., 2000; Rabinkov et al., 2000), S-allylmercaptocysteine (CSSA) and S-allylmercaptocaptopril (CPSSA) (Miron et al., 2004) possess antioxidant and SH-modifying activities similar to those of allicin, although milder. In particular, it was disclosed that a 16 h allicin treatment of N87 cells (a human gastric adenocarcinoma cell line) and CB2 cells (a Chinese hamster ovary cell line) inhibited DNA synthesis and cell proliferation in a dose-response manner (Miron et al., 2003), and that allicin induced apoptosis in B-CLL cells (Arditti et al., 2005).

As shown in Example 1 hereinafter, SA-6MP and SA-6MPR have been readily prepared by reacting 6-MP or 6-MPR, respectively, with allicin, in an aqueous solvent, as depicted in Scheme 1 hereinafter.

The reaction is preferably performed under mild basic conditions, e.g., in the presence of a buffer having a basic pH in the range of 7.2-8.5. The process of preparing the compound of the present invention may further comprise isolating the obtained compound from the reaction mixture, and optionally a purification of the obtained compound by any of the purification methods known. As particularly described in Example 1, the isolation of the compound from the reaction mixture was effected by collecting the precipitate formed upon cooling the reaction mixture; and purification of the precipitate was performed by recrystallization from a water:ethanol mixture.

Compounds according to the present invention can also be prepared from the acetyleno (propargyl) analog of allicin, dipropargyldithiosulfinate, as it performs the same thiolation reaction.

The compounds of the present invention may further be prepared by any other preparative methods used for the preparation of unsymmetrical disulfides as described by Antoniow and Witt (2007).

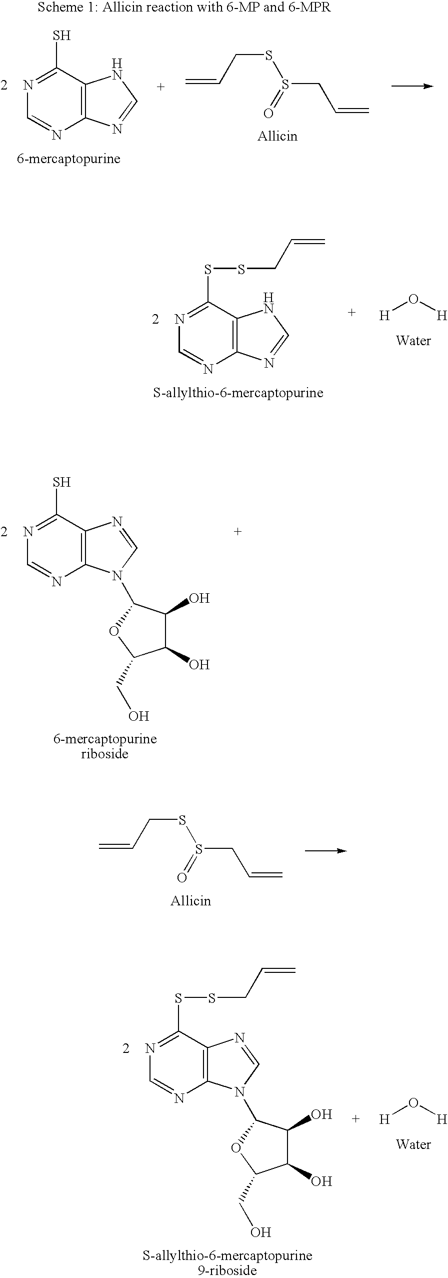

As shown in Example 2, both SA-6MP and SA-6MPR were found to act as highly efficient anti-proliferative agents. In particular, both compounds have been shown to have DNA synthesis inhibitory effect, which was either equal or superior to that of the non-conjugated components, namely, 6-MP, 6-MPR and allicin. Furthermore, while treating various cancer cells with these compounds, an increased lethal effect and a significant increase in the percentage of apoptotic B-CLL cells were observed in cells treated with SA-6MP, as compared with cancer cells treated with the non-conjugated mercaptopurine 6-MP. The high efficacy of the compounds of the present invention to induce apoptosis in B-CLL cells indicates that these compounds are highly efficient as therapeutic agents for the treatment of leukemia. The therapeutic efficacy of these compounds was further reflected by the inhibition of the metabolic activity of various cancer cells, which was found to be about 90% for a range of cancer cells.

In summary, two new 6-MP analogs, SA-6MP and SA-6MPR were synthesized and characterized. The biological effects of these new prodrugs on several cancer cell lines were assessed and the IC₅₀ values obtained from the XTT assay represent the susceptibility of cells to SA-6MP and SA-6MPR. While the various types of leukemia cells showed a high sensitivity to the new drugs, adhesive cell lines were less sensitive.

The biological effects of SA-6MP and SA-6MPR on cell viability and proliferation were compared to those of the parent reactants and were found to be concentration dependent. Both SA-6MP and SA-6MPR were found to exert more potent deleterious effects than those of the original prodrugs on all the cell lines tested, while there was only a slight difference between the antiproliferative activities of SA-6MP and SA-6MPR, in favor of SA-6MP, in most cell lines. However, MOLT4 and Jurkat cells were more sensitive to 6-MPR than to 6-MP, compared to the other cell lines tested. In view of the fact that 6-MP-resistant MOLT4 cells exhibited enhanced sensitivity to methylmercaptopurine riboside (meMPR) (Fotoohi et al., 2006), suggesting the existence of a distinct transport route for meMPR and the bypass of that of 6-MP, a possible explanation for the “inverted” sensitivity may dwell in 6-MP resistance mechanisms. The resistant cells exhibited significant reduction in levels of mRNA encoding several proteins involved in the de novo purine synthesis, as well as in levels of ribonucleoside triphosphates, as compared to non-resistant cells.

It might have been expected that the combined activity of the 6-mercaptopurines and allicin would increase the antiproliferative potential of the new derivatives, as compared with each parental component, but it did not exceed the antiproliferative activity of allicin, possibly due to the dual potency of the allicin molecule. It did, however, improve the antiproliferative properties of 6-MP and 6-MPR.

The increased potency of SA-6MP and SA-6MPR, as compared to the parent prodrugs, namely 6-MP and 6-MPR, respectively, can be attributed to 3 mechanism of action:

(i) The combined properties of both moieties, i.e. the mercaptopurine, a nucleotide analog that interferes with nucleic acid synthesis, and the allylmercapto residue derived from allicin, that causes depletion of reduced glutathione and other essential free SH groups in the cell, thereby leading to apoptosis (Miron et al., 2007). Both effects were shown to be exerted by the new prodrugs; inhibition of DNA synthesis in Daudi, Hela and N87 cells, and an increased number of apoptotic B-CLL cells;

(ii) Higher hydrophobicity of the new prodrugs enables better penetration into the cells, as compared with the parent molecules. Consequently, larger amounts of 6-MP or 6-MPR are released from the intracellular reaction between glutathione and the allylthio-prodrugs; and

(iii) 6-MP and 6-MPR have a free SH, which upon oxidation, forms an inactive dimer connected by an S—S bridge (purine-S—S-purine) (Goyal et al., 2001). Nonactive 6-MP dimers were indeed found in the medium of 6-MP treated cell lines but not in the medium of SA-6MP treated cells. The fact that only a fraction of the thiopurine molecules occurs in its active form explains the need for a high prodrug concentration. The allylthio moiety of the new derivative protects the free SH from such oxidation. Only later, upon entry to the cellular reducing environment is the mixed disulfide SA-6MP cleaved, which renders higher efficiency at lower concentrations. The proposed mechanism for the reaction of SA-6MP with free thiols in the cell is depicted in Scheme 2 hereinafter.

The highest antiproliferative activity of all the compounds tested was exerted by allicin (Miron et al., 2003, 2007; Arditti et al., 2005). Since targeted killing by allicin production in situ is a complex procedure (Miron et al., 2003; Arditti et al., 2005) and other means of administration suffer drawbacks, its combination with 6-MP and 6-MPR is the most feasible treatment at present.

In view of the aforesaid it is concluded that the compounds of the present invention can be efficiently and beneficially utilized in the treatment of medical conditions that are treatable by mercaptopurines. Furthermore, in cases wherein the compound of the present invention is a purine residue linked via a disulfide bond to an allyl group, the compounds of the present invention can further be utilized in the treatment of medical conditions that are treatable by allicin.

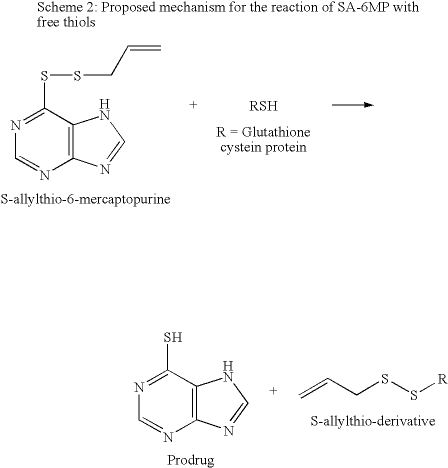

In another aspect, the present invention thus relates to a pharmaceutical composition comprising a compound as defined above, or a pharmaceutically acceptable salt thereof, and a pharmaceutical acceptable carrier.

Examples of medical conditions that are currently known as treatable by mercaptopurines include, without being limited to, proliferative diseases and disorders, particularly leukemia, inflammatory diseases and disorders, skin diseases and disorders and immune diseases and disorders. Examples of medical conditions that are currently known as treatable by allicin include, without being limited to, cardiovascular diseases and cardiovascular risk factors, hypertension, proliferative diseases and disorders such as cancer, bacterial infections, viral infections, parasitic infections and fungal infections.

Thus, in one embodiment, the pharmaceutical composition of the present invention is for treatment of a disease or disorder selected from a proliferative disease or disorder, an inflammatory disease or disorder, a skin disease or disorder, or an immune disease or disorder. Preferred compounds for such uses are compounds wherein R₂ is a covalent bond, R₁ and R₃ each is H, R₄ is H or a riboside residue, and Y is allyl, namely SA-6MP or SA-6MPR, respectively, or pharmaceutically acceptable salts thereof.

The term “proliferative disease or disorder” as used herein refers to a disease or disorder characterized by enhanced cell proliferation. Cell proliferation conditions which may be prevented or treated by the present invention include, for example, malignant tumors such as cancer and benign tumors.

The proliferative disease or disorder that can be treated with the compounds of the present invention may be cancer such as brain cancers such as glioblastoma multiforme, anaplastic astrocytoma, astrocytoma, ependyoma, oligodendroglioma, medulloblastoma, meningioma, sarcoma, hemangioblastoma, and pineal parenchymal; skin cancers such as melanoma and Kaposi's sarcoma; papilloma, blastoglioma, ovarian cancer, prostate cancer, squamous cell carcinoma, astrocytoma, head cancer, neck cancer, bladder cancer, breast cancer, lung cancer, colorectal cancer, thyroid cancer, pancreatic cancer, gastric cancer, hepatocellular carcinoma, leukemia, lymphoma, Hodgkin's lymphoma and Burkitt's lymphoma.

As shown in the Examples section hereinafter, both SA-6MP and SA-6MPR were found to be highly effective in inhibiting the growth and/or killing cancerous blood cells, thus, these compounds are particularly effective for the treatment of leukemia.

Other non cancerous proliferative disorders are also treatable using the compounds of the present invention. Such non cancerous proliferative disorders include, for example, stenosis, restenosis, in-stent stenosis, vascular graft restenosis, arthritis, rheumatoid arthritis, diabetic retinopathy, angiogenesis, pulmonary fibrosis, hepatic cirrhosis, atherosclerosis, glomerulonephritis, diabetic nephropathy, thrombic microangiopathy syndromes and transplant rejection.

The inflammatory disease or disorder that can be treated with the compounds of the present invention includes, for example, polymyositis, septic/toxic shock, acute respiratory distress syndrome (ARDS), asthma, systemic lupus erythematosus, dermatitis (contact hypersensitivity), peritoneal inflammation, delayed-type hypersensitivity reactions, reperfusion injury, burn injury, transplant rejection, chronic inflammatory disease, hemorragic-traumatic shock, metastases, etc. In particular, the conjugates described herein are useful for the treatment of chronic inflammatory diseases and disorders including, but not limited to, inflammatory bowel diseases such as Crohn's disease and ulcerative colitis, Behcet's disease, polymyositis, Reiter's syndrome, psoriatic arthritis, systemic lupus erythematosus and vasculitis.

The skin disease or disorder that can be treated with the compounds of the present invention includes, but is not limited to, psoriasis, atopical dermatitis, contact dermatitis and further eczematous dermatitises, seborrhoeic dermatitis, Lichen planus, Pemphigus, bullous Pemphigoid, Epidermolysis bullosa, urticaria, angioedemas, vasculitides, erythemas, cutaneous eosinophilias, Lupus erythematosus and acne. The compounds described herein are in particular useful for the treatment of psoriasis.

The term “immune disease or disorder” as used herein refers to any disease or disorder associated with a development of an immune reaction, either a cellular or a humoral immune reaction, or both, and/or which affects the immune system. Examples of immune diseases and disorders include inflammatory diseases and disorders, allergy and autoimmune diseases.

Non-limiting examples of immune diseases include demyelinating diseases that are characterized by a demyelinating process of the central nervous system, such as, e.g., multiple sclerosis, sub-acute sclerosing panencephalomyelitis (SSPE), metachromatic leukodystrophy, inflammatory demyelinating polyradiculoneuropathy, Pelizaeus-Merzbacher disease and Guillain-Barré syndrome.

The term “autoimmune disease” generally relates to an immune disease wherein the immune response is developed against antigens normally present in the affected patient, for example an organ specific autoimmune disease (an immune response specifically directed against for example, the endocrine system, the hematopoietic system, the skin, the cardiopulmonary system, the neuromuscular system, the central nervous system, etc) or a systemic autoimmune disease, e.g., Systemic lupus erythematosous, Rheumatoid arthritis, polymyositis, transplant rejection etc. The compounds described herein are in particular useful for the treatment of autoimmune diseases or disorders such as polymyositis, systemic lupus erythematosus or rejection following organ transplants.

The pharmaceutical composition provided by the present invention may be prepared by conventional techniques, e.g., as described in Remington: The Science and Practice of Pharmacy, 19th Ed., 1995. The composition may be in solid, semisolid or liquid form and may further include pharmaceutically acceptable fillers, carriers or diluents, and other inert ingredients and excipients. Furthermore, the pharmaceutical composition can be designed for a slow release of the conjugate. The composition can be administered by any suitable route, e.g. intravenously, orally, parenterally, rectally, or transdermally. The dosage will depend on the state of the patient, and will be determined as deemed appropriate by the practitioner.

The route of administration may be any route which effectively transports the active compound to the appropriate or desired site of action, the oral route being preferred. If a solid carrier is used for oral administration, the preparation may be tabletted, placed in a hard gelatin capsule in powder or pellet form or it can be in the form of a lozenge. If a liquid carrier is used, the preparation may be in the form of a syrup, emulsion or soft gelatin capsule. Tablets, dragees or capsules having talc and/or a carbohydrate carrier or binder or the like are particularly suitable for oral application. Preferable carriers for tablets, dragees or capsules include lactose, corn starch and/or potato starch.

Thus, in still another aspect, the present invention relates to use of a compound as defined above, or a pharmaceutically acceptable salt thereof, for the treatment of a disease or disorder selected from a proliferative disease or disorder, an inflammatory disease or disorder, a skin disease or disorder, or an immune disease or disorder.

In a further aspect, the present invention provides a method for treatment of a disease or disorder selected from a proliferative disease or disorder, an inflammatory disease or disorder, a skin disease or disorder, or an immune disease or disorder, said method comprising administering to an individual in need a therapeutically effective amount of a compound as defined above or a pharmaceutically acceptable salt thereof.

Preferred compounds for use in the method of the present invention are SA-6MP and SA-6MPR, or pharmaceutically acceptable salts thereof.

The invention will now be illustrated by the following non-limiting Examples.

### EXAMPLES

### Materials and Methods

Materials and General Methods 2,3-bis(2-methoxy-4-nitro-5-sulfophenyl)-5-[(phenylamino)carbonyl]-2H-tetrazolium hydroxide (XTT), 6-mercaptopurine, 6-mercaptopurine riboside, deuterocholoroform (CDCl₃), phenazine methosulfate (PMS) and propidium iodine (PI) were obtained from Sigma (St Louis, Mo.). [Methyl-³H] thymidine was purchased from Amersham (UK).

Alliin was synthesized as previously described (Stoll and Seebeck, 1951). Allicin was produced by applying synthetic alliin on an immobilized alliinase column (Miron et al., 2006), and the concentration was determined by HPLC as previously described (Miron et al., 2002). 2-nitro 5-thio-benzoic acid (NTB) was prepared as previously described (Miron et al., 1998).

Mass spectra were recorded on a Micromass Platform LCZ 4000 Mass Spectrometer Instrument, using ESI-Electro Spray Ionization Mode, at the following conditions: samples were directly infused at 5 μl/minute, maintaining the nitrogen flow at 360 liters/hour; the capillary used was 4.16 KV; the cone voltage was 43 V, and the extractor voltage was 4 V; the source block temperature was kept at 100° C. and the desolvation temperature was kept at 150° C.; LM RES 14.4; HM RES 14.4; and Ion Energy 0.5. NMR experiments were performed on a Bruker Avance-500 spectrometer. SA-6MP and SA-6MPR were dissolved in CDCl₃ at about 5-10 mM. Their complete assignments were determined using a combination of 1D (¹H, ¹³C, DEPT) and 2D (gs-COSY, gs-HSQC) NMR experiments. HPLC analyses of 6-MP derivatives were done on a LiChrosorb RP-18 (7 μm) column, using methanol (60%) in water containing 0.01% trifluoroacetic acid, at a flow rate of 0.55 ml/min, and their absorbance was detected at 210 nm. The concentration of the pure S-allylthio-6-mercaptopurine derivatives was also determined with NTB (Miron et al., 1998) using ε_{M} 14150 M⁻¹cm⁻¹ at 412 nm.

### Cell Culture, Cell Viability and Apoptosis Assay

The following cell lines were used: N87, a human gastric adenocarcinoma cell line; Hela HtTA-1 cells, a human cervix carcinoma cell line, clone HtTA-1 (Gossen and Bujard, 1992) and MDR HT-29, a human colon adenocarcinoma cell line. These cells were grown in monolayers, using Dulbecco modified Eagle's medium (DMEM) supplemented with antibiotics and 10% heat-inactivated fetal calf serum (FCS); All the other cells were grown in suspension: among them were established cell lines such as HL60, a human leukocyte promyelocytic leukemia; U937, human myelomonocytic cells; MOLT4, a T-lymphoblastic cell line derived from acute lymphoblastic leukemia; Jurkat, a human T cell, lymphoblast-like cell; Daudi, a B-lymphoblastoid cell line derived from Burkitt lymphoma. B-CLL, peripheral blood mononuclear cells (PBMC) were obtained from heparinized whole blood drawn from patients at Rai stage IV with their written consent. Blood cells were subjected to ficoll density gradient centrifugation and the mononuclear cells were diluted to the desired concentration. The cells in suspensions were maintained in RPMI-1640 supplemented with 2 mM L-glutamine, antibiotics and 10% (v/v) heat-inactivated fetal calf serum (FCS).

Cell proliferation was determined by the XTT viability assay in 96-well plates, based on the reduction of tetrazolium salt to soluble formazan compounds by living cells. Cells (10,000-15,000 cells/well) were seeded in a 96-well plate. After 16 h incubation with various concentrations of 6-MP, 6-MPR and their S-allylthio-derivatives, 50 μl of XTT/PMS mixture (50 μM PMS, 0.1% XTT in medium) was added onto the cells. After an incubation period of 3-4 h at 37° C. the absorbance of the samples was measured in an ELISA Reader at 450 nm. SDS (1%, 10 μl/well) was added to reference wells before adding the XTT/PMS solution.

The effects of 6-MP derivatives on DNA synthesis were assessed by [Methyl-³H] thymidine incorporation into DNA. All the experiments performed with cells were carried out at least in triplicates. Adhesive cells (N87, Hela HtTA-1 and MDR HT-29) were seeded at 10,000 cells/well (96-well plate) or 60,000 cells/well (24-well plate). Cells in suspension (B-CLL, Daudi, HL-60, Jurkat, MOLT4 and U937cells) were seeded at 15,000 cells/well (96-wells plate) or 100,000 cells/well (24-wells plate). Adhesive cells were grown at 37° C. for 6 h after seeding, before treatment. For the assessment of [Methyl-³H] thymidine incorporation, cells were treated with various concentrations of 6-MP derivatives at 37° C. for 16 h in the presence of [Methyl-³H] thymidine (0.8-1.0 μCi/well). Then, plates were frozen (−20° C., 1 h). Adhesive cells were trypsinized before harvesting. Cells in suspension were directly harvested after thawing out the frozen cells.

Apoptosis analysis in B-CLL cells treated with 6-MP derivatives at different concentration (16 h, at 37° C.) was done by FACS analysis. B-CLL cells were incubated with FITC-CD19 anti-human antibodies (Becton Dickinson, NJ, USA) for 20 minutes at 4° C. After washing off the unbound antibodies, samples were incubated with 5 μl Annexin-Cy5 (Pharmingen, San Diego, Calif., USA) in 10 mM HEPES pH 7.4 buffer containing 140 mM NaCl and 2.5 mM CaCl₂, (HBS) for 10 minutes at room temperature. Subsequently, unbound Annexin was washed out and the samples were analyzed using FACScan analyzer (Becton-Dickinson, NJ, USA). The lymphocytes were counted and gated according to their size in forward and side scatters.

Cell death was monitored by trypan blue dye exclusion test or propidium iodide (PI) incorporation. Treated cells were incubated with PI (2 μg/ml) for 20 min at 37° C., washed with HBS and examined by fluorescence microscopy or analyzed by flow cytometry using fluorescence-activated cell sorting (Becton Dickinson FACScan Instrument using CellQuest software (BD Bioscience, San Jose, Calif.). Monolayer cells were trypsinized and washed with HBS before FACS analysis.

### Statistical Analysis

The results of viability and proliferation were expressed as mean values±SD (n=3-6). For each cell line, the results were analyzed using two-way analysis of variance (ANOVA) followed by Bonferroni's posttest for the factors of the drugs used and their various concentrations, considering p<0.05 as significant. IC₅₀ values (mean±SEM) were obtained from the linear range of the viability curve versus drug concentration (XTT assay).

### Example 1

### Synthesis of S-allylthio-6-mercaptopurine (SA-6MP) and S-allylthio-6-mercaptopurine 9-riboside (SA-6MPR)

S-allylthio-6-mercaptopurine (SA-6MP) was prepared by reacting 6-mercaptopurine (6-MP) and allicin, as depicted in Scheme 1 above. A solution of 6-MP (1 mmole) in ethanol (100 ml) was added at room temperature to allicin (0.55 mmole) in aqueous solution (55 ml). The pH was adjusted to 8.0-8.4 using solid NaHCO₃ to 0.025M (final concentration). The reaction rate was monitored by HPLC analysis until 6-MP was no longer detected (about 10 hours). Ethanol was partially removed by rotaevaporation and the slightly turbid solution was stored at 4° C. The product, SA-6MP, which crystallized, was collected by filtration, washed with cold water and dried. A second harvest was done after removal of ethanol from the filtrate and storage at 4° C. for precipitation. The overall yield was 80%. Re-crystallization was done after re-dissolving the precipitate in ethanol and adding water.

S-allylthio-6-mercaptopurine 9-riboside (SA-6MPR) was prepared by reacting 6-mercaptopurine riboside (6-MPR) and allicin, as depicted in Scheme 1 above. A solution of 6-MPR (0.6 mmole), in 0.04 M phosphate buffer, pH 7.2 (40 ml), was added at room temperature to a solution of allicin (0.35 mmole), in 50% ethanol (10 ml). The reaction proceeded for 4 hours and was stored at 4° C. The reaction rate was monitored by HPLC analysis. The product, a white precipitate, was harvested by filtration. A second harvest was done after removal of ethanol and storage at 4° C. The overall yield was 85%. Re-crystallization was done as described above.

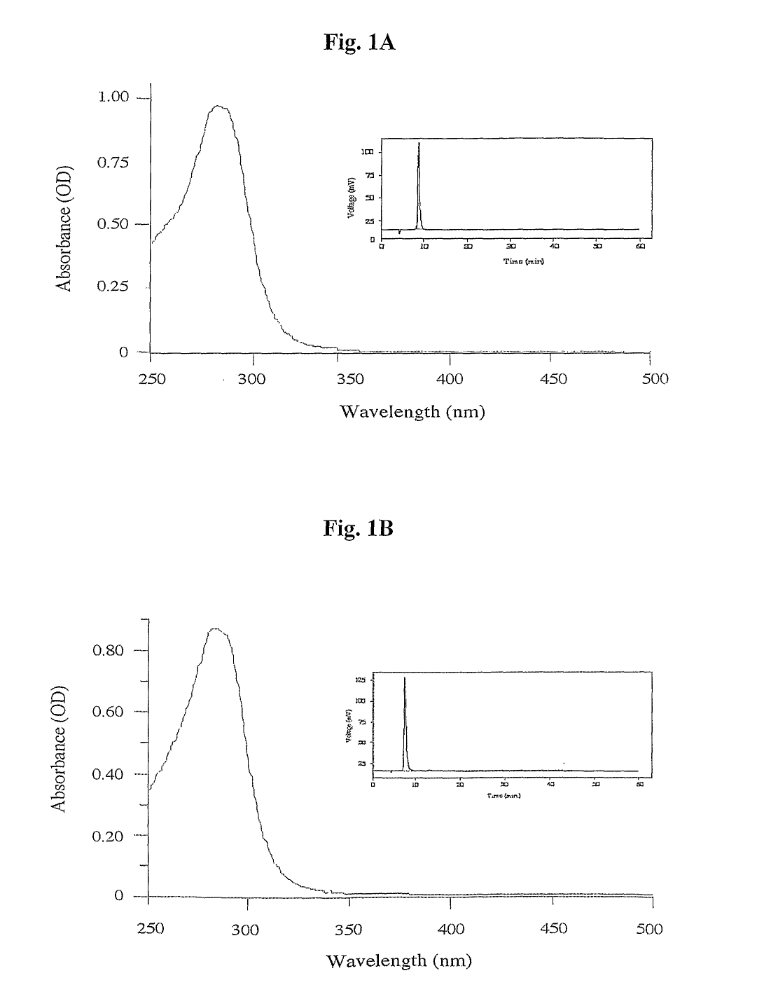

The synthesis of both SA-6MP and SA-6MPR were confirmed by mass spectroscopy analysis (electrospray ionization, ESI) and NMR. SA-6MP (molecular weight 224) is an off white crystal, showing a maximum absorbance in ethanol at 283 nm, E^{M}₂₈₃ was 13,780 M⁻¹cm⁻¹. ESI-MS: m/z (%)=[M+H]⁺=225.2 (40); DMSO=79 (100). SA-6MPR appeared as white crystals (ESI-MS: molecular weight 356), its maximum absorbance in ethanol at 284 nm E^{M}₂₈₄ was 14,240 M⁻¹cm⁻¹. HPLC retention times for SA-6MP and SA-6MPR were 8.7 and 7.3 min, respectively, as shown in FIGS. 1A-1B. NMR analysis is shown in Table 1 hereinbelow. The ClogP values (hydrophobicity partition coefficient) were: 6-MP: 0.823; SA-6MP: 1.344; 6-MPR: −1.191; SA-6MPR 0.90.

TABLE 1
¹H and ¹³C NMR chemical shifts
of SA-6MP and SA-6MPR in CDCl₃

| | |
|---|---|
| SA-6MP | SA-6MPR |

| No. | H (ppm) | | C (ppm) | H (ppm) | | C (ppm) |
|---|---|---|---|---|---|---|
| 1 | 12.19 | (s) | | | | |
| 2 | | | 131.10 | | | 132.53 |
| 3 | | | 160.46 | | | 161.40 |
| 5 | 8.96 | (s) | 152.11 | 8.78 | (s) | 151.60 |
| 7 | | | 149.36 | | | 147.29 |
| 9 | 8.30 | (s) | 141.47 | 8.10 | (s) | 143.64 |
| 12 | 3.61 | (d) | 41.76 | 3.56 | (d) | 41.65 |
| 13 | 5.91 | (m) | 132.00 | 5.87 | (m) | 131.84 |
| 14 | 5.14 | (m) | 119.63 | 5.12 | (m) | 119.80 |
| 15 | | | | 5.88 | (d) | 91.5 |
| 17 | | | | 4.39 | (s) | 87.72 |
| 18 | | | | 3.90 | (dd) | 63.09 |
| 19 | | | | 4.5 | (d) | 72.39 |
| 20 | | | | 5.12 | (m) | 73.74 |

### Example 2

### The Biological Activity of SA-6MP and SA-6MPR on Cell Lines

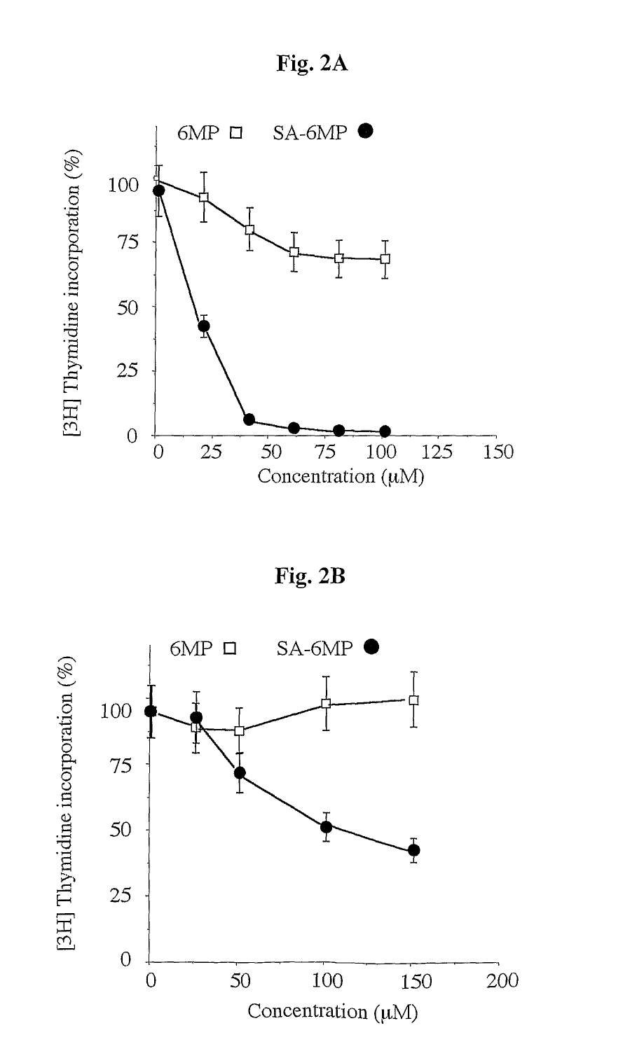

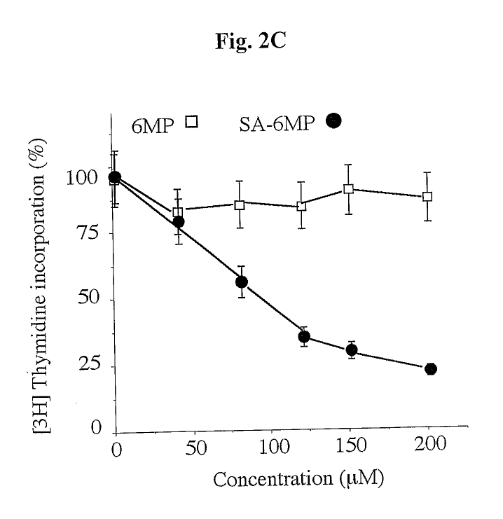

The antiproliferative effect of 6-MP and SA-6MP on various cell lines was assessed by determining [³H] thymidine incorporation into the DNA. 6-MP and SA-6MP at 0-200 μM were applied to Daudi, Hela and N87 cells cultured in 96-well plates in the presence of [³H] thymidine, as described in Materials and Methods. As shown in FIGS. 2A-2C, SA-6MP inhibited DNA synthesis at a much higher efficacy than 6-MP and in all the cell lines tested, treatment resulted in a dose-dependent inhibition of cell proliferation. As further shown, the sensitivity to the prodrug is cell type dependent. Thus, in SA-6MP-treated Daudi cells, 50% inhibition was observed at about 20 μM, compared to 100 μM in N87 cells and 110 μM in Hela cells. 6-MP caused no inhibition of proliferation, at the same concentrations.

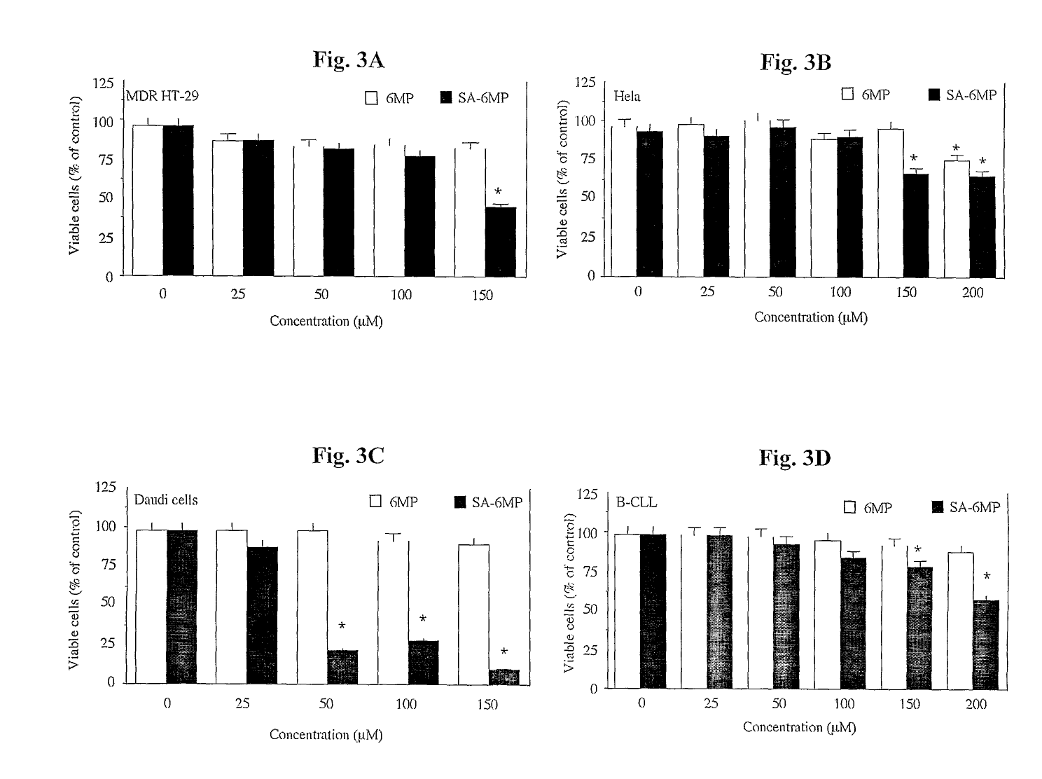

In parallel, the trypan blue dye exclusion test was used to assess cell death. In particular, different concentrations of 6MP and SA-6MP were applied to various cell cultures (Daudi and Hela cells, each at 30,000 cells/well) as described in Materials and Methods. The cells were stained with trypan blue to monitor the lethal effect of several concentrations of the tested conjugates as the percentage of dead cells. As shown in FIG. 3A-3D, cell death induced by 6-MP and SA-6MP was both concentration and cell type dependent. In particular, monolayer cell lines such as MDR HT-29 and Hela cells were almost non-sensitive to 6-MP and SA-6MP treatment for 16 h at 100 μM; however, both cell cultures treated with 150 μM SA-6MP showed a reduced viability, 40% and 65% respectively, compared to non-treated cells (FIGS. 3A-3B, respectively). Treatment of Hela cells with 6-MP at 200 μM resulted in slightly reduced viability (75%). In Daudi cells treated with SA-6MP at 50 μM, the residual viability after 16 h was about 20%, whereas 6-MP at concentrations higher than 100 μM showed no significant effect on cell viability (FIG. 3C). B-CLL cells were almost insensitive to 6-MP treatment (100-200 μM, 16 h). SA-6MP at concentrations higher than 150 μM reduced the residual viability to 75% (FIG. 3D).

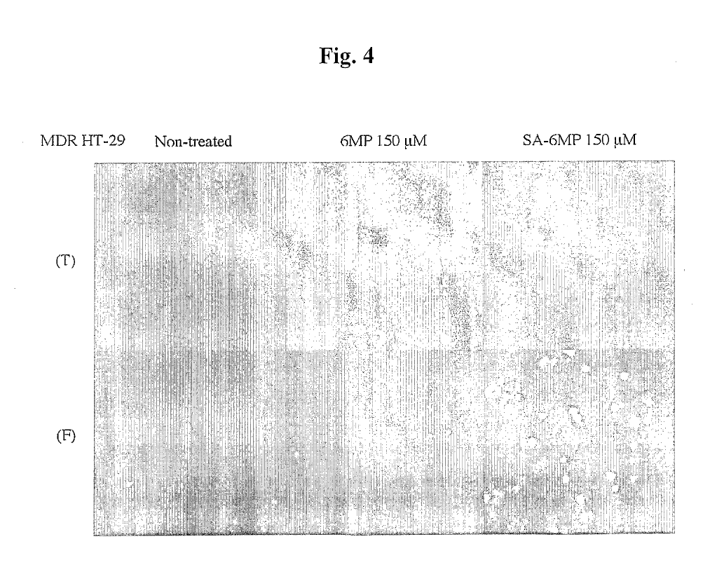

In order to determine the toxic effect of 6-MP and SA-6MP on multidrug resistant cell lines, MDR HT-29 cells were treated for 16 h with 150 μM of either 6-MP or SA-6MP for 16 h, and were then stained with PI. As can be observed from the phase microscopy results shown in FIG. 4, upper panel, epithelial-like growth was inhibited in cells treated with SA-6MP, whereas no inhibition was observed for 6-MP treatment. This finding was further supported by fluorescence microscopy of the cells stained with PI, shown in FIG. 4, lower panel, indicating that the inhibition resulted in cell death.

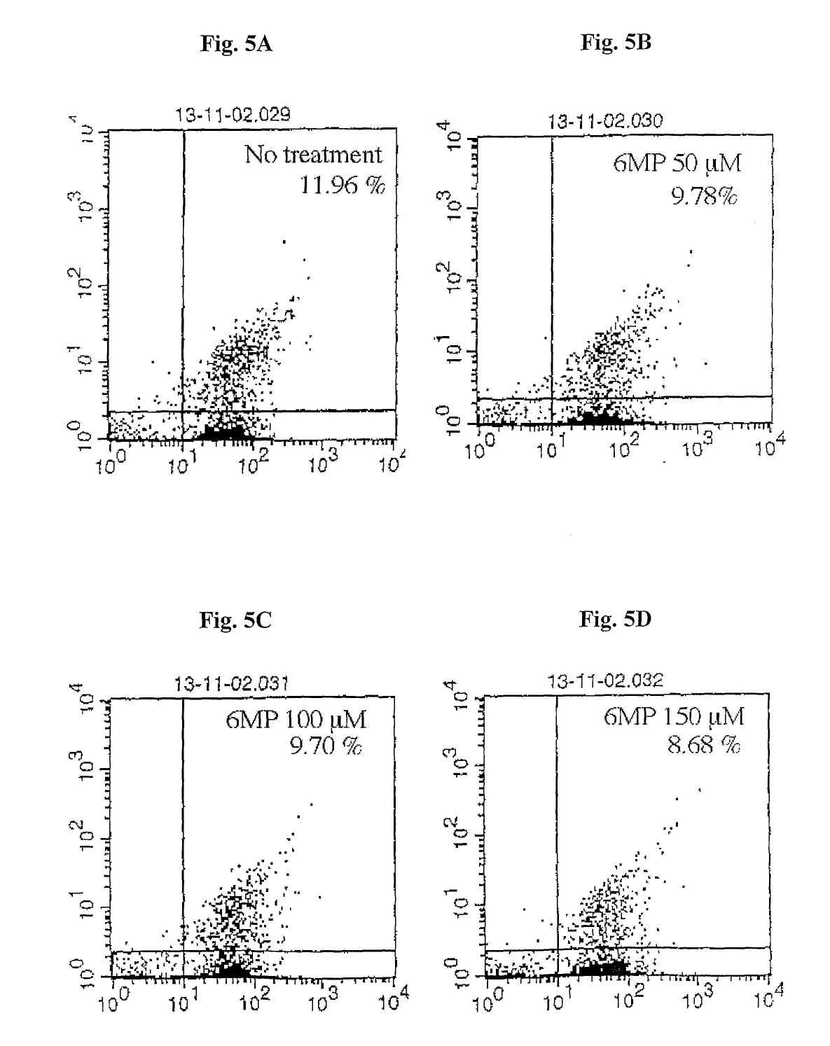

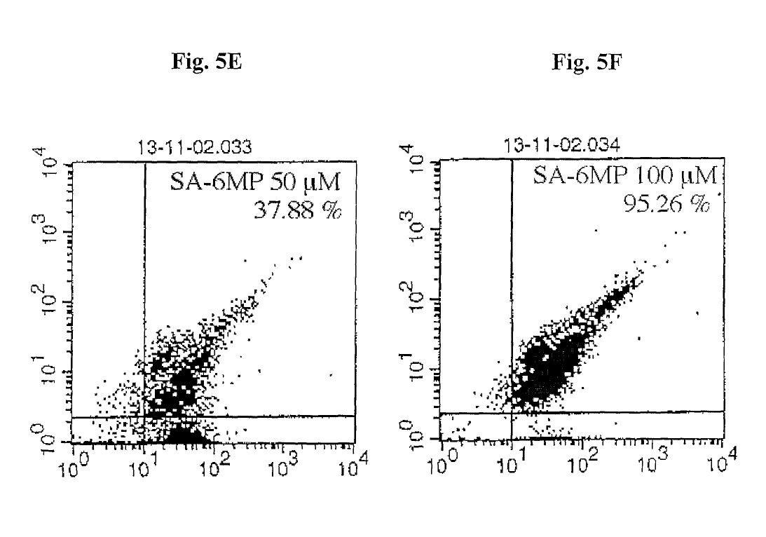

The slight decrease in viability of B-CLL human peripheral blood mononuclear cells (PBMC) upon incubation in the presence of 6-MP or SA-6MP was further investigated. B-CLL cells were treated with various concentrations of either 6-MP or SA-6MP and were subjected to flow cytometry analysis, as described in Materials and Methods, in order to monitor cells undergoing apoptosis. Sorting showed that there was no significant increase in the percent of apoptotic cells treated for 16 h with 6-MP at the range between 0-150 μM (FIGS. 5A-5D); however, when following treatment with 50 and 100 μM SA-6MP, the percent of apoptotic cells increased to 38 and 95%, respectively (FIGS. 5E-5F).

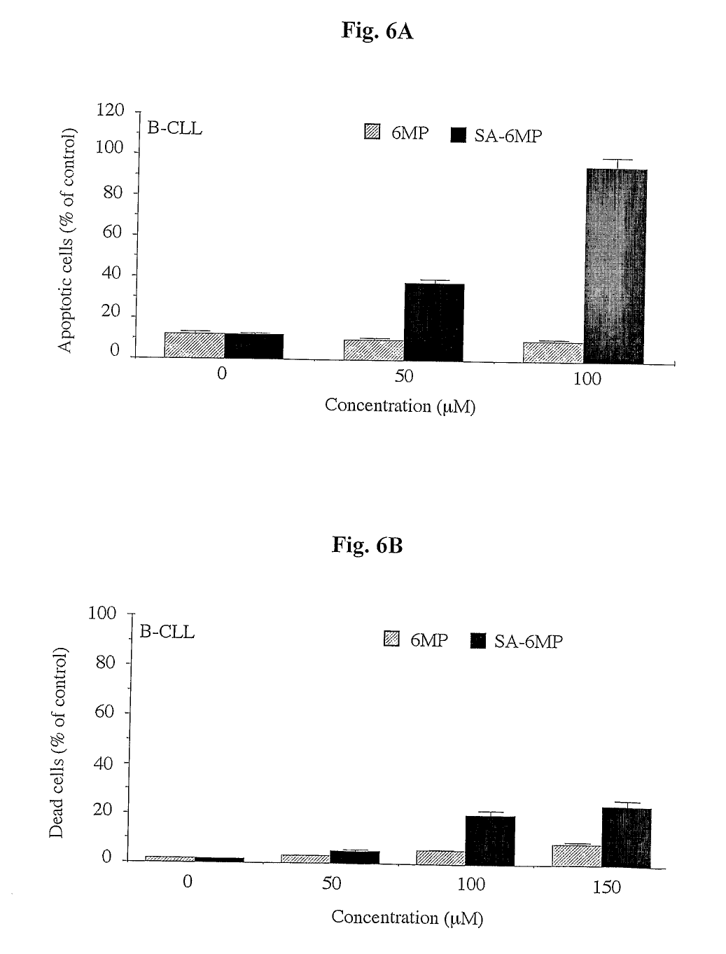

As shown in FIGS. 6A-6B, apoptosis (FACS analysis with annexin) and cell death (trypan blue test) induced in B-CLL by 6-MP and SA-6MP did not occur simultaneously.

The inhibitory effects of the various prodrugs on Daudi leukemia cell and on monolayer N87 cells were compared. Cells were seeded in 96-well plates and were then incubated for 16 h at 37° C. with 0-150 μM of 6MP, 6MPR, SA-6MP or SA-6MPR. XTT was added to the wells for 3 h at 37° C. Cell viability was monitored using an ELISA reader at 450 nm, as described in Materials and Methods.

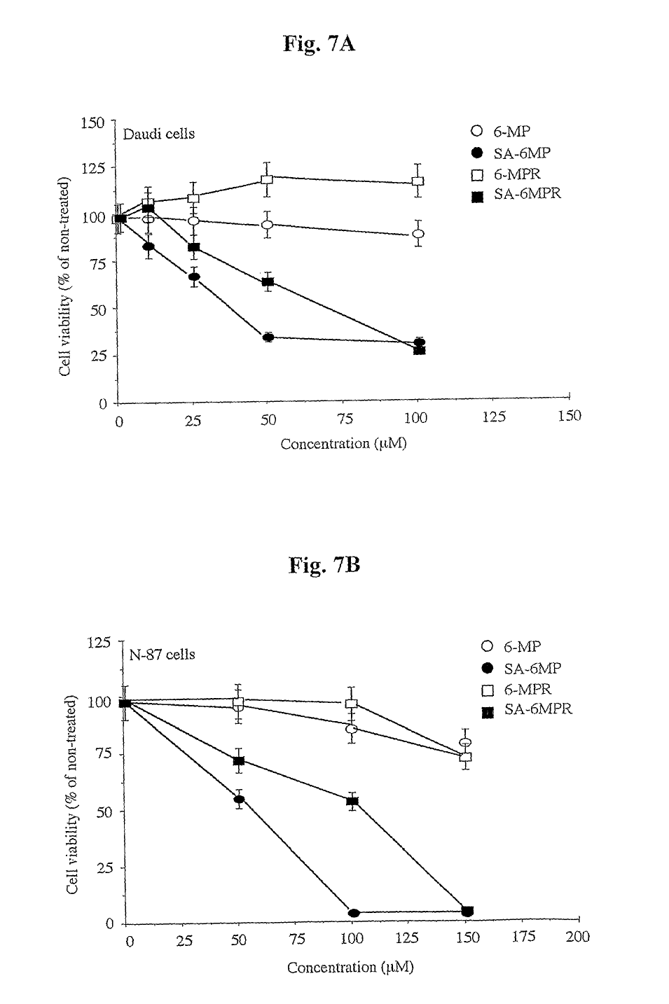

As shown in FIGS. 7A-7B, Daudi cells and N87 cells grown in the presence of 6-MP or 6-MPR (0-100 μM) showed no significant loss of cell proliferation. A slightly decreased proliferation was observed for N87 cells at 150 μM (6-MP˜80%; 6-MPR˜75%, p<0.05). Contrary to 6-MP and to 6-MPR, SA-6MP had a very potent anti-proliferative effect on Daudi cells, reducing their proliferation to 15-30% at 50 μM. In N87 cells treated with the same concentration, the residual proliferation was 50-60%. Treatment with SA-6MPR showed similar results. The residual proliferation of Daudi cells-treated with SA-6MPR at 50 or 100 μM was 60% and 25%, respectively, whereas in N87 cells treated with SA-6MPR at 50 or 100 μM, the residual viability was about 70% and 55%, respectively. There was a complete loss of proliferation in N87 cells treated with SA-6MPR at 150 μM.

The concentration effects of 6-MP, 6-MPR SA-6MP and SA-6MPR on cell proliferation (IC₅₀ values) were used to assess the efficacy of the different drugs in various cell lines. In particular, cell in suspension, namely, Daudi, HL-60, U937, Molt-4, Jurkat and B-CLL, were tested at prodrug concentration of 0-100 μM, and monolayer cells, namely, Hela HtTA-1, MDR HT-29 and N87, were tested at prodrug concentration of 0-200 μM. in both cases, cell viability was determined by trypan blue dye exclusion assay. Data are presented in Table 2 hereinbelow for 6MP derivatives-treated for 16 h.

TABLE 2
Anti-proferative concentrations of various 6-MP derivatives on cancer cell lines

| | Anti-proferative concentration - IC₅₀ (mean ± SE) in μM | | | | |
|---|---|---|---|---|---|
| Cell line | 6MP | SA-6MP | 6MPR | SA-6MPR | Allicin |
| Daudi | >200 | 38.3 ± 3.9 | ^{a} | 70.8 ± 7.8 | 35.3 ± 4.2 |
| HL-60 | ^{a} | 42.0 ± 3.8 | ^{b} | 86.1 ± 9.5 | 5.5 ± 0.6 |
| U937 | >200 | 46.7 ± 4.8 | >200 | 112.6 ± 10.9 | 19.7 ± 2.1 |
| Molt-4 | 50.5 ± 6.1 | 8.9 ± 1.2 | 24.8 ± 2.5 | 16.9 ± 2.3 | ^{c} |
| Jurkat | >200 | 54.7 ± 6.3 | 99.0 ± 11.9 | 69.1 ± 7.5 | ^{c} |
| B-CLL ^{a} | >900 | 129.1 ± 14.2 | ^{c} | ^{c} | ^{c} |
| Hela | >200 | 114.4 ± 12.0 | ^{b} | >200 | 103.5 ± 10.2 |
| MDR HT-29 | >200 | 126.3 ± 19.1 | ^{a} | >200 | 83.8 ± 7.8 |
| N87 | ^{a} | 70.5 ± 8.5 | ^{a} | 120.0 ± 15.6 | 16.8 ± 3.3 |

^{a} No change in viability was detected
^{b} Viability of treated cells increase (+20%) of non-treated
^{c} Not determined

As shown in Table 2, the new derivatives, SA-6MP and SA-6MPR, were found to be much more effective in inducing cytotoxicity than the parent drugs, 6-MP, 6-MPR. Furthermore, SA-6MP was a better agent than SA-6MPR. In particular, among the leukemia cell lines treated, the most sensitive was Molt-4 cell line. It is noteworthy that Molt-4 and Jurkat cells, both T cell leukemia cell lines, were more sensitive to the 6-MPR than to 6-MP compared to the other cell lines tested. The sensitivity of the leukemia cell lines Daudi, HL-60 and U937 to treatment with either SA-6MP or SA-6MPR was similar. B-CLL cells from human peripheral blood mononuclear cells (PBMC) were highly resistant to the treatment. Nevertheless, the FACS results, indicating apoptotic processes in progress (FIGS. 5E-5F), suggest that longer incubation times may result in cell death. The monolayer cell lines tested were less sensitive to SA-6MP and SA-6MPR than cells in suspension.

As shown in Table 2, the calculated IC₅₀ of 6-MP for most of the cell lines tested was higher than 200 μM under a 16 h exposure to the drug. This might seem a rather high concentration, as compared with other results reported in the nM range. As previously disclosed by Sugiyama et al. (2003), showing the in vitro effect of 6-MP on T-cell mitogen-induced blastogenesis of human peripheral blood mononuclear cells (PBMCs), the IC₅₀ values after 4 days of treatment with azathioprine (AZ) or 6-MP were 230.4±231.3 and 149.5±124.9 nM, respectively. As the treated cells in that work were different from those used in this case, and so was the period of exposure, it is impossible to compare the results disclosed by Sugiyama et al. with the results presented in Table 2. However, when B-CLL cells were treated with allicin for 48 h, the calculated IC₅₀ for allicin induced-apoptosis was 20 nM, whereas exposure of allicin for only 16 h resulted in IC₅₀ which was approximately 1000 times higher (Arditti et al., 2005, FIG. 1).

### Example 3

### The Efficacy of Mercaptopurine Derivatives in Treatment of Colitis

In this experiment, the anti-inflammatory activity of the compounds of the present invention in examined in vivo, using mice in which chronic inflammation of the colon has been induced as a model for colitis. In particular, the chronic inflammation is induced either by intrarectal administration of 2,4,6-trinitrobenzene sulfonic acid (TNBS) (80 mg/kg body weight, dissolved in 0.9% NaCl, 30 μl/mouse) or by administration of dextran sodium sulphate (DSS, 2% w/v) in the drinking water for 5 days.

Mice are treated with various amounts of either 6-MP or a compound of the present invention, or with a control vehicle, and the development of inflammation is followed 7-13 days after the induction of colitis. Development of Colitis is assessed daily by measurement of body weight and stool consistency. At the end of the experiment, mice are sacrificed by cervical dislocation. The colon length and the histological parameters are used to evaluate the efficacy of the various treatments. Macroscopic lesion analysis is performed as previously described (Wallace et al., 1989).

### REFERENCES

Abramovitz D., Gavri S., Harats D., Levkovitz H., Mirelman D., Miron T., Eilat-Adar S., Rabinkov A., Wilchek M., Eldar M., Vered Z., Coron. Artery. Dis., 1999, 10, 515-519
Agarwal K. C., Med. Res. Rev., 1996, 16, 111-124
Antoniow S., Witt D., A novel and efficient synthesis of unsymmetrical disulfides, Synthesis, 2007, 3, 363-366
Arditti F. D., Rabinkov A., Miron T., Reisner Y., Berrebi A., Wilchek M., Mirelman D., Molecular Cancer Therapeutics, 2005, 4, 325-331
Cara C. J., Pena A. S., Sans M., Rodrigo L., Guerrero-Esteo M., Hinojosa J., Garcia-Paredes J., Guijarro L. G., Med. Sci. Monit., 2004, 10, RA247-RA254
Carroll W. L., Bhojwani D., Min D. J., Raetz E., Relling M., Davies S., Downing J. R., Willman C. L., Reed J. C., Hematology, 2003, 102-131
Coulthard S., Hogarth L., Invest New Drugs, 2005, 23, 523-532
Dubinsky M. C., Clin Gastroenterol Hepatol, 2004, 2, 731-743
Eilat S., Oestraicher Y., Rabinkov A., Ohad D., Mirelman D., Battler A., Eldar M., Vered Z., Coronary Artery Dis, 1995, 6, 985-990
Elion G. B., Burgi E., Hitchings G. H., J. Am. Chem. Soc. 1952, 74, 411-414
Elkayam A., Mirelman D., Peleg E., Wilchek M., Miron T., Rabinkov A., Sadetzki S., Rosenthal T., Am. J. of Hypertension, 2000, 14, 377-381
Fotoohi A. K., Wrabel A., Moshfegh A., Peterson C., Albertioni F., Biochem. Pharmacol., 2006, 72, 816-823
Freeman F., Kodera Y., J. Agricultural and Food Chem., 1995, 43, 2332-2338
Gonen A., Harats D., Rabinkov A., Miron T., Mirelman D., Wilchek M., Weiner L., Ulman E., Levkovitz H., Ben-Shushan D., Shaish A., Pathobiology, 2005, 72, 325-334
Gossen M., Bujard H., Proc. Natl. Acad. Sci. USA, 1992, 89, 5547-5551
Goyal R. N., Rastogi A., Sangal A., New J. Chem., 2001, 25, 545-550
Hirsch K., Danilenko M., Giat J., Miron T., Rabinkov A., Wilchek M., Mirelman D., Levy J., Sharoni Y., Nutrition and Cancer, 2000, 38, 245-254
Karran P., Br Med Bull, 2006, 79-80, 153-170
Koch H. P., Lawson L. D., Garlic: The Science and therapeutic application of Allium sativum l. and related species, Williams & Wilkins, Baltimore, 1996
Krynetski E. Y., Evans W. E., Pharm Res, 1999, 16, 342-349
Lachmann G., Lorenz D., Radeck W., Steiper M., Arzneimittelforschung, 1994, 44, 734-743
Lang A., Lahav M., Sakhnini E., Barshack I., Fidder H., Avidan B., Bardan E., Hershkoviz R., Bar-Meir S., Chowers Y., Clinical Nutrition, 2004, 23, 1199-1208
Miron T., Rabinkov A., Mirelman D., Weiner L., Wilchek M., Anal. Biochem., 1998, 265, 317-325
Miron T., Rabinkov A., Mirelman D., Wilchek M., Weiner L., Biochim. Biophys. Acta, 2000, 1463, 20-30
Miron T., Shin I., Feigenblat G., Weiner L., Mirelman D., Wilchek M., Rabinkov A., Anal. Biochem., 2002, 307, 76-83
Miron T., Mironchik M., Mirelman D., Wilchek M., Rabinkov A., Mol. Cancer. Ther., 2003, 2, 1295-1301
Miron T., Rabinkov A., Peleg E., Rosenthal T., Mirelman D., Wilchek M., Am. J. Hypertens., 2004, 17, 71-73
Miron T., SivaRaman H., Rabinkov A., Mirelman D., Wilchek M., Anal. Biochem., 2006, 351, 152-154
Miron T., Wilchek M., Sharp A., Nakagawa Y., Naoi M., Nozawa Y., Akao Y., J Nutr. Biochem., 2007 [Talia: Please provide the full reference, if exists]
Rabinkov A., Miron T., Konstantinovski L., Wilchek M., Mirelman D., Weiner L., Biochim. Biophys. Acta, 1998, 1379, 233-244
Rabinkov A., Miron T., Mirelman D., Wilchek M., Glozman S., Yavin E., Weiner L., Biochim. Biophys. Acta, 2000, 1499, 144-153
Stoll A., Seebeck E., Adv. Enzymol., 1951, 11, 377-400
Sugiyama K., Satoh H., Hirano T., J Pharm Pharmacol., 2003, 55, 393-398
Wallace J. L., MacNaughton W. K., Morris G. P., Beck P. L., Inhibition of leukotriene synthesis markedly accelerates healing in a rat model of inflammatory bowel disease, Gastroenterology, 1989, 96, 29-36
Watters J. W., McLeod H. L., Biochim Biophys Acta, 2003, 1603, 99-111

1. A compound of the general formula
X—S—S—Y
wherein X is a purine residue of the general formula: 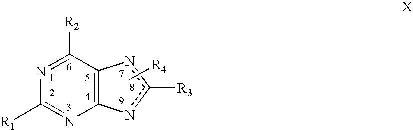
wherein
R₁ to R₃ each independently is either a covalent bond or selected from the group consisting of H, halogen, SH, NR₅R₆, O-hydrocarbyl, S-hydrocarbyl, heteroaryl, unsubstituted hydrocarbyl, hydrocarbyl substituted by halogen, CN, SCN, NO₂, OR₅, SR₅, NR₅R₆ or heteroaryl, and a carbohydrate residue, wherein R₅ and R₆ each independently is H or hydrocarbyl or R₅ and R₆ together with the nitrogen atom to which they are attached form a 5- or 6-membered saturated heterocyclic ring optionally containing 1-2 further heteroatoms selected from the group consisting of oxygen, nitrogen and sulfur, the additional nitrogen being unsubstituted or substituted by alkyl substituted by halogen, hydroxyl or phenyl, provided that one of R₁, R₂ and R₃ is a covalent bond;
R₄ is H, alkyl, a carbohydrate residue or NR₅R₆, wherein R₅ and R₆ each independently is H or hydrocarbyl or R₅ and R₆ together with the nitrogen atom to which they are attached form a 5- or 6-membered saturated heterocyclic ring optionally containing 1-2 further heteroatoms selected from the group consisting of oxygen, nitrogen and sulfur, the additional nitrogen being unsubstituted or substituted by alkyl substituted by halogen, hydroxyl or phenyl;
Y is a linear or branched, cyclic or acyclic, radical selected from the group consisting of C₇-C₂₀ alkenyl, C₂-C₂₀ alkynyl, C₃-C₂₀ cycloalkyl and C₃-C₂₀ cycloalkenyl, wherein said radical is heteroaryl, unsubstituted hydrocarbyl, or hydrocarbyl substituted by halogen, CN, SCN, NO₂, OR₇, SR₇, NR₇R₈ or heteroaryl, wherein R₇ and R₈ each independently is H or hydrocarbyl or R₇ and R₈ together with the nitrogen atom to which they are attached form a 5- or 6-membered saturated heterocyclic ring optionally containing 1-2 further heteroatoms selected from the group consisting of oxygen, nitrogen and sulfur, the additional nitrogen being unsubstituted or substituted by alkyl substituted by halogen, hydroxyl or phenyl; and
the dashed line denotes a double bond between the carbon atom at position 8 and either the nitrogen atom at position 7 or the nitrogen atom at position 9, provided that, when the double bond is between the carbon atom at position 8 and the nitrogen atom at position 7, R4 is at position 9, and when the double bond is between the carbon atom at position 8 and the nitrogen atom at position 9, R4 is at position 7; and
“hydrocarbyl” means a saturated or unsaturated, linear or branched, cyclic or acyclic, or aromatic radical selected from the group consisting of C₁-C₂₀ alkyl, C₂-C₂₀ alkenyl, C₂-C₂₀ alkynyl, C₃-C₂₀ cycloalkyl, C₃-C₂₀ cycloalkenyl, C₆-C₁₄ aryl, (C₁-C₂₀)alkyl(C₆-C₁₄)aryl, and (C₆-C₁₄) aryl(C₁-C₂₀)alkyl;
“heteroaryl” means a radical derived from a mono- or poly-cyclic heteroaromatic ring containing 1 to 3 heteroatoms selected from the group consisting of O, S and N;
and pharmaceutically acceptable salts thereof.

2. The compound of claim 1, wherein one of R₁ to R₃ is a covalent bond and the other two of R₁ to R₃ each independently is H, SH, halogen, hydrocarbyl, O-hydrocarbyl or S-hydrocarbyl, wherein the hydrocarbyl is C₁-C₆ alkyl, or NR₅R₆, wherein R₅ and R₆ each independently is H or C₁-C₆ alkyl or R₅ and R₆ together with the nitrogen atom to which they are attached form a 6-membered saturated heterocyclic ring optionally containing one further heteroatom selected from the group consisting of oxygen, nitrogen and sulfur, R₄ is H or a carbohydrate residue, and Y is hydrocarbyl.

3. The compound of claim 2, wherein one of R₁ to R₃ is a covalent bond and the other two of R₁ to R₃ each independently is H, chloro, SH, C₁-C₆ alkyl, or —NR₅R₆, wherein R₅ and R₆ each independently is H or C₁-C₆ alkyl, R₄ is H or a monosaccharide residue, and Y is C₂-C₆ alkenyl or alkynyl.

4. The compound of claim 3, wherein said monosaccharide residue is a 5- or 6-membered monosaccharide residue.

5. The compound of claim 3, wherein one of R₁ to R₃ is a covalent bond and the other two of R₁ to R₃ each independently is H, chloro, SH, methyl, or dimethylamino, R₄ is H or a riboside residue, and Y is C₃ alkenyl or alkynyl.

6. The compound of claim 1, wherein (i) R₂ is a covalent bond; (ii) R₁ is a covalent bond; or (iii) R₃ is a covalent bond.

7-8. (canceled)

9. The compound of claim 6, wherein (i) R₂ is a covalent bond, R₁ is H, SH, methyl or dimethylamino, R₃ is H, SH or methyl, R₄ is H or a 5-membered monosaccharide residue, and Y is allyl or propargyl; (ii) R₁ is a covalent bond, R₂ is H, SH, methyl or dimethylamino, R₃ is H, SH or methyl, R₄ is H or a 5-membered monosaccharide residue, and Y is allyl or propargyl; or (iii) R₃ is a covalent bond, R₁ and R₂ each independently is H, SH, methyl or dimethylamino, R₄ is H or a 5-membered monosaccharide residue, and Y is allyl or propargyl.

10-11. (canceled)

12. The compound of claim 9, wherein (i) R₂ is a covalent bond, R₁ and R₃ each is H, R₄ is H or a riboside residue, and Y is allyl or propargyl; (ii) R₁ is a covalent bond, R₂ and R₃ each is H, R₄ is H or a riboside residue, and Y is allyl or propargyl; or (iii) R₃ is a covalent bond, R₁ and R₂ each is H, R₄ is H or a riboside residue, and Y is allyl or propargyl.

13-14. (canceled)

15. The compound of claim 12, S-allylthio-6-mercaptopurine (herein designated SA-6MP) wherein R₂ is a covalent bond, R₁, R₃ and R₄ each is H, and Y is allyl, and pharmaceutically acceptable salts thereof.

16. The compound of claim 12, S-allylthio-6-mercaptopurine 9-riboside (herein designated SA-6MPR) wherein R₂ is a covalent bond, R₁ and R₃ each is H, R₄ is a riboside residue, and Y is allyl, and pharmaceutically acceptable salts thereof.

17. A pharmaceutical composition comprising a compound of claim 1 or a pharmaceutically acceptable salt thereof, and a pharmaceutical acceptable carrier.

18. The pharmaceutical composition of claim 17, for treatment of a disease or disorder selected from the group consisting of a proliferative disease or disorder, an inflammatory disease or disorder, a skin disease or disorder, and an immune disease or disorder.

19. The pharmaceutical composition of claim 18, wherein said proliferative disease or disorder is cancer; said inflammatory disease or disorder is selected from the group consisting of Crohn's disease, ulcerative colitis, Behcet's disease, polymyositis, Reiter's syndrome, psoriatic arthritis, systemic lupus erythematosus and vasculitis; said skin disease or disorder is psoriasis; and said immune disease or disorder is an autoimmune disease or disorder selected from the group consisting of polymyositis, systemic lupus erythematosus and rejection following organ transplants.

20. The pharmaceutical composition of claim 19, wherein said cancer is leukemia.

21. (canceled)

22. The pharmaceutical composition of claim 17, wherein said compound is S-allylthio-6-mercaptopurine or S-allylthio-6-mercaptopurine 9-riboside.

23. (canceled)

24. A method for treatment of a disease or disorder selected from the group consisting of a proliferative disease or disorder, an inflammatory disease or disorder, a skin disease or disorder, and an immune disease or disorder, said method comprising administering to an individual in need a therapeutically effective amount of a compound of claim 1 or a pharmaceutically acceptable salt thereof.

25. The method of claim 24, wherein said proliferative disease or disorder is cancer; said inflammatory disease or disorder is selected from the group consisting of Crohn's disease, ulcerative colitis, Behcet's disease, polymyositis, Reiter's syndrome, psoriatic arthritis, systemic lupus erythematosus and vasculitis; said skin disease or disorder is psoriasis; and said immune disease or disorder is an autoimmune disease or disorder selected from the group consisting of polymyositis, systemic lupus erythematosus, and rejection following organ transplants.

26. The method of claim 25, wherein said cancer is leukemia.

27. (canceled)
